(19) United States
(12) Patent Application Publication
(10) Pub. No.: US 2026/0042972 A1
(43) Pub. Date: Feb. 12, 2026

(54) MIXED COMPONENT TRACTION FLUIDS

(71) Applicant: VANTAGE SANTOLUBES RESEARCH LLC, New Hope, PA (US)

(72) Inventors: Salvatore Michael PERNA, Greer, SC (US); Thomas Reginald FORBUS, Spartanburg, SC (US)

(21) Appl. No.: 19/101,209

(22) PCT Filed: Aug. 4, 2023
(86) PCT No.: PCT/US2023/071713

§ 371 Date: Feb. 4, 2025

Related U.S. Application Data
(60) Provisional application No. 63/370,595, filed on Aug. 5, 2022.

(51) Int. Cl. C10M 105/04; C10M 129/10; C10M 133/04; C10N 10/04; C10N 30/02; C10N 30/04; C10N 30/06; C10N 30/10; C10N 30/18; C10N 40/04

(52) U.S. Cl. CPC C10M 105/04; C10M 129/10; C10M 133/04; C10M 2203/045; C10M 2207/023; C10M 2215/02; C10N 2010/04; C10N 2030/02; C10N 2030/04; C10N 2030/06; C10N 2030/10; C10N 2030/18; C10N 2040/046

(57) ABSTRACT

A lubricating fluid includes a blend of at least two isomers of hydrogenated linear AMS dimers, one of which is a hydrogenated linear AMS dimer having a formula of 2,4-dicyclohexyl-2-methylpentane and another of which is a hydrogenated linear AMS dimer (HLD) isomer having a formula of 1,4-dicyclohexyl-4-methylpentane. The blend reduces viscosities at low temperatures (0° C. and lower), without severely lowering high shear stress EHD shear strength (or traction coefficients), and further inhibits foaming issues which are typical in mixtures of very different fluids such as silicones and hydrocarbons combinations.

### CROSS-REFERENCE TO RELATED APPLICATIONS

This application claims priority to U.S. Provisional Patent Application No. 63/370,595, filed Aug. 5, 2022, the entire disclosure of which is hereby incorporated herein by reference.

### TECHNICAL FIELD

The present teachings relate generally to traction fluids, and more specifically to lubricating base oil mixtures providing high elastohydrodynamic (EHD) shear strength traction fluids with improved low temperature and minimal impact on the high EHD traction coefficients.

### BACKGROUND

An infinitely variable speed transmission (traction drive) fluid has similar responsibilities to normal (geared) transmission (i.e., automatic transmission) fluids in that it must serve as a lubricant, coolant, and in some cases, hydraulic fluid. A traction or infinitely variable transmission (IVT) fluid has the added responsibility of transmitting torque from an input device to the output through the lubricating film that it forms in the contact(s) between smooth rolling-sliding rotating elements of the transmission. Thus, the fluid is required to exhibit high shear strength in the high shear stress EHD conditions found in the area of contact between the rolling-sliding drive elements which are separated and lubricated by a thin film of the IVT fluid. The fluid's resistance to shear (shear strength) in the contact provides the torque transmitting capability of the fluid composition.

Lubricating fluids suitable for use in infinitely variable transmissions for most equipment employed in outdoor applications need a critical balance of good low temperature flow properties and high EHD shear strength, and particularly high shear strength at a combination of high temperatures and low contact stresses. Cycloaliphatic hydrocarbons generally have superior shear strength properties, but very poor low temperature properties compared to more typical hydrocarbon-based fluids employed for producing good lubricants. Other fluids having good low temperature properties can be incorporated, which may improve the low temperature properties of the mixture but generally these cause significant undesirable loss of the shear strength properties. It has now been found that traction fluids comprising 2,4-dicyclohexyl-2-methylpentane (HLD), derived from perhydrogenation of the linear dimer of alpha-methylstyrene (AMS), as the base oil, have high EHD shear strength (or traction coefficients). In sub-ambient temperatures, traction performance decreases due to high fluid viscosities under those operating conditions.

A favorable method was found in US 2007/0057226 whereby low viscosity polydimethylsiloxane fluids was incorporated with HLD to improve low temperature flow of the traction fluid with only very minimal impact on the outstanding torque transfer capability of the fluid relative to fluid produced with only HLD. However, such mixtures with polydimethylsiloxanes suffer from foaming issues when operating at high speeds with unavoidable air entrainment. These foaming issues limit the utility of this formulation approach in various applications. Other traction fluids are disclosed in US 2020/0347314, US 2019/0177249, US 2015/0105305, US 2010/0048434, and US 2007/0063170. However, even with these known fluids, there is still a need for improvement with respect to low temperature flow properties.

Thus, there exists a need for improved traction fluids that address the above problem as well as other drawbacks.

### SUMMARY

The needs set forth herein as well as further and other needs and advantages are addressed by the present embodiments, which illustrate solutions and advantages described below.

It is an object of the present teachings to provide base oil mixtures for lubricants to maintain elastohydrodynamic (EHD) shear strength and excellent low temperature performance while providing suitable foaming performance at high speeds.

These and other objects of the present teachings are achieved by providing base oil mixtures comprising a blend of at least two isomers of hydrogenated linear alpha-methylstyrene (AMS) dimers. In particular, the present teachings provide a lubricating fluid comprising 2,4-dicyclohexyl-2-methylpentane (HLD) having the formula:

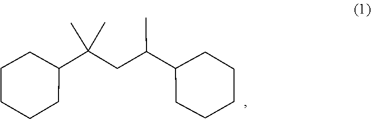 [SMILES: CC(CC(C)(C)C1CCCCC1)C1CCCCC1]

and 1,4-dicyclohexyl-4-methylpentane (HLD isomer) having the formula:

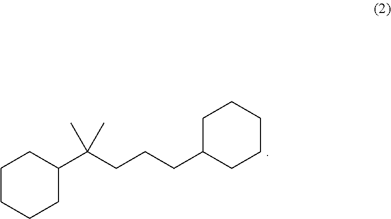 [SMILES: CC(C)(CCCC1CCCCC1)C1CCCCC1]

Other features and aspects of the present teachings will become apparent from the following detailed description, taken in conjunction with the accompanying drawings, which illustrate by way of example the features in accordance with embodiments of the present teachings. The summary is not intended to limit the scope of the present teachings, which is defined by the claims included herein.

### BRIEF DESCRIPTION OF THE DRAWINGS

FIG. 1 illustrates a manner of using the lubricating fluid according to the present teachings.

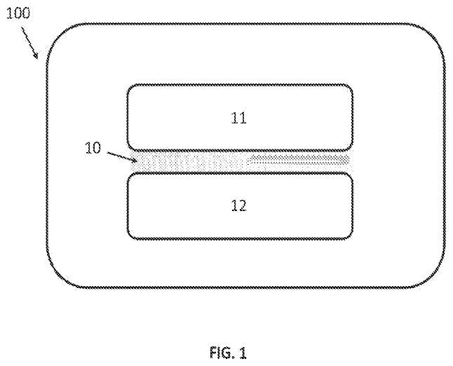

FIG. 2 illustrates the relationship between Dynamic Viscosity at −30° C. and the HLD Isomer weight percentage of a lubricating fluid according to the present teachings.

### DETAILED DESCRIPTION

The present teachings are described more fully hereinafter with reference to the accompanying drawings, in which the present embodiments are shown. The following description illustrates the present teachings by way of example, not by way of limitation of the principles of the present teachings.

The present teachings have been described in language more or less specific as to structural features. It is to be understood, however, that the present teachings are not limited to the specific features shown and described, since the product and/or method herein disclosed comprises preferred forms of putting the present teachings into effect.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. Other than in the operating examples, or where otherwise indicated, all numbers expressing quantities of ingredients or reaction conditions used herein are to be understood as modified in all instances by the term “about”.

The present teachings provide base oil mixtures suitable for producing high elastohydrodynamic (EHD) shear strength traction fluids with improved low temperature performance and minimal impact on the high EHD traction coefficients relative to pure (96+weight %) HLD base oils.

### Base Oils

The present teachings provide a blend of two isomers of hydrogenated linear AMS dimers to enable the production of lubricants that maintain high elastohydrodynamic (EHD) shear strength and excellent low temperature performance while providing suitable foaming performance at high speeds.

The present teachings utilize mixtures comprising a Hydrogenated Linear AMS Dimer (HLD) with the structure of formula (1):

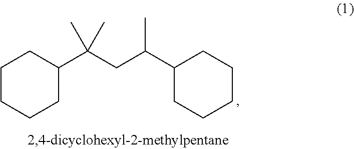 [SMILES: CC(CC(C)(C)C1CCCCC1)C1CCCCC1]

and a Hydrogenated Linear AMS Dimer (HLD) isomer with the structure of formula (2):

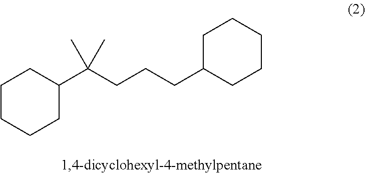 [SMILES: CC(C)(CCCC1CCCCC1)C1CCCCC1]

### Characterizations of Base Oils

The lubricating fluids of the present teachings may be characterized by a variety of standard tests known to one of ordinary skill in the art. The energy efficiency of the lubricating fluids may be affected by the viscosity of the lubricating fluids and the traction coefficient of the lubricating fluids. The viscosity of the lubricating fluids is closely related to its ability to reduce friction in the contacts between solid surfaces. The traction coefficient of the lubricating fluids is related to the energy losses with a certain load.

Traction coefficients may be measured using PCS Mini-Traction Machine (MTM) from PCS Instruments, Ltd. measured at various slide-to-roll ratios (e.g., 0.1% to 200%), temperatures, and loads ranging from 20N to 70N or a maximum Hertzian contact stress of 0.5 GPa to 1.5 GPa.

Viscosity, often referred to as Dynamic Viscosity (DV), can be measured using Kinematic Viscosity (KV). Kinematic Viscosity (KV) may be determined by ASTM D445-06 Standard Test Method for Kinematic Viscosity of Transparent and Opaque Liquids (and Calculation of Dynamic Viscosity). Kinematic Viscosity may also be calculated from a measurement of Dynamic Viscosity (DV) at low shear rates and density using the equation:

$$K V = \frac{D V}{ρ}$$

where ρ is density.

Viscosity Index (VI) is a unitless measurement of a lubricating fluid's change in viscosity relative to temperature change. The higher the VI, the more stable the viscosity remains over temperature fluctuations. Viscosity Index may be determined by ASTM D2270-04 Standard Practice for Calculating Viscosity Index from Kinematic Viscosity at 40° C. and 100° C.

The following Table 1 illustrates a wide range of blending of the two isomers of perhydrogenated linear AMS dimers.

In some embodiments of the base oil mixture, the HLD-to-IsoHLD ratio range of 5:1 to 2.4:1 produces blends with a low trough where dynamic viscosities at sub-zero temperatures are minimized. The base oil mixture comprises about 81.9 weight % to about 69.4 weight % of the Hydrogenated Linear AMS Dimer (HLD); and about 16.4 weight % to about 28.9 weight % of the Hydrogenated Linear AMS Dimer (HLD) isomer. Shear strength measurements comparing “Pure” HLD (Fluid 1) with the mixed fluid #8 show that Fluid #8 (HLD-to-IsoHLD mixture) with a 52% lower dynamic viscosity (29,497 vs 14,097 cP, respectively) at −30° C., maintains maximum traction coefficients of 90-93% to those of “pure” HLD (Fluid 1) with steel-on-steel specimens loaded to a maximum Hertzian Contact Stress of 1.2 GPa throughout the temperature range of 40-120° C. measured at 1.0 meters/sec entrainment velocity. Further, Fluid #8 has better low temperature dynamic viscosity and would have high traction coefficients relative to Fluid #9, which has similar low temperature dynamic viscosity.

In other embodiments of the base oil mixture, the HLD-to-IsoHLD ratio range of 8:1 to 1.5:1 produces blends with a low trough where dynamic viscosities at sub-zero temperatures are minimized. The base oil mixture comprises about 87.5 weight % to about 59.3 weight % of the Hydrogenated Linear AMS Dimer (HLD); and about 10.8 weight % to 39.1 weight % of the Hydrogenated Linear AMS Dimer (HLD) isomer. Shear strength measurements comparing “Pure” HLD (Fluid 1) with the mixed fluid #6 show that Fluid #6 (HLD-to-IsoHLD mixture) with a 31% lower dynamic viscosity (29,497 vs 20,246 cP, respectively) at −30° C.

In still other embodiments of the base oil mixture, the HLD-to-IsoHLD ratio range of 10:1 to 1.3:1 produces blends with a low trough where dynamic viscosities at sub-zero temperatures are minimized. The base oil mixture comprises about 89.8 weight % to 55.4 weight % of the Hydrogenated Linear AMS Dimer (HLD); and about 8.5 weight % to 42.9 weight % of the Hydrogenated Linear AMS Dimer (HLD) isomer. Shear strength measurements comparing “Pure” HLD (Fluid #1) with the mixed fluid #7 show that Fluid #7 (HLD-to-IsoHLD mixture) with a 36% lower dynamic viscosity (29,497 vs 18,784 cP, respectively) at −30° C. Shear strength measurements comparing “Pure” HLD (Fluid #1) with the mixed fluid #5 show that Fluid #5 (HLD-to-IsoHLD mixture) with a 25% lower dynamic viscosity (29,497 vs 22,065 cP, respectively) at −30° C.

TABLE 1
Composition and Properties of Hydrogenated Linear Dimer Base Fluids

| Fluid # | | | | | | | | | | |
|---|---|---|---|---|---|---|---|---|---|---|
| 1 | 2 | 3 | 4 | 5 | 6 | 7 | 8 | 9 | 10 | 11 |

Lubricating Fluid Composition (weight %)

| | | | | | | | | | | | |
|---|---|---|---|---|---|---|---|---|---|---|---|
| HLD | 96.5 | 94.3 | 92.0 | 90.9 | 89.8 | 87.5 | 85.3 | 81.9 | 69.4 | 59.3 | 55.4 |
| HLD Isomer | 1.8 | 4.0 | 6.3 | 7.4 | 8.5 | 10.8 | 13.0 | 16.4 | 28.9 | 39.1 | 42.9 |
| “HLD” Total | 98.3 | 98.3 | 98.3 | 98.3 | 98.3 | 98.3 | 98.3 | 98.3 | 98.3 | 98.4 | 98.3 |
| HLD:Isomer | 54:1 | 24:1 | 15:1 | 12:1 | 10:1 | 8:1 | 6.5:1 | 5:1 | 2.4:1 | 1.5:1 | 1.3:1 |
| Ratio | | | | | | | | | | | |

Kinematic Viscosity (cSt)

| | | | | | | | | | | | |
|---|---|---|---|---|---|---|---|---|---|---|---|
| 40° C. | 20.7 | 20.3 | 19.9 | 19.7 | 19.6 | 19.2 | 18.9 | 17.7 | 16.6 | 16.1 | 15.4 |
| 100° C. | 3.65 | 3.61 | 3.56 | 3.53 | 3.54 | 3.50 | 3.47 | 3.35 | 3.24 | 3.19 | 3.10 |
| VI | 18 | 18 | 17 | 15 | 19 | 20 | 17 | 23 | 23 | 25 | 25 |

Low Temperature Dynamic Viscosity (cP)

| | | | | | | | | | | | |
|---|---|---|---|---|---|---|---|---|---|---|---|
| 0° C. | 324 | 268 | 255 | 224 | 239 | 234 | 228 | 200 | 193 | 217 | 233 |
| −10° C. | 1003 | 868 | 822 | 796 | 783 | 738 | 708 | 599 | 618 | 674 | 692 |
| −20° C. | 4621 | 3992 | 3408 | 3595 | 3142 | 3107 | 2970 | 2445 | 2429 | 2669 | 2815 |
| −30° C. | 29479 | 25474 | 22655 | 22810 | 22065 | 20246 | 18784 | 14097 | 14005 | 15828 | 16221 |

Traction Coefficient

| | | |
|---|---|---|
| 40° C. | 0.121 | 0.113 |
| 60° C. | 0.114 | 0.103 |
| 80° C. | 0.104 | 0.093 |
| 100° C. | 0.092 | 0.083 |
| 120° C. | 0.083 | 0.075 |

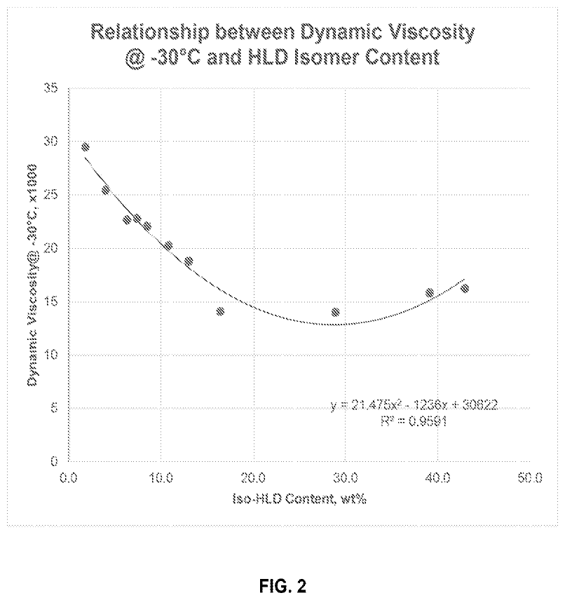

As shown in FIG. 2, when HLD isomer is at about 30 weight % and HLD is at about 70 weight %, the base oil mixture has an advantageously low dynamic viscosity at −30° C.

### Additives

The various embodiments of lubricating fluids according to the present teachings may further comprise at least one additive that in some embodiments may be selected from the group consisting of: dispersant, detergent, defoamer, antioxidant, rust inhibitor, friction modifier, corrosion inhibitor, extreme pressure additive, anti-wear additive, pour point depressant, and combinations thereof.

Examples of the dispersant, including ashless dispersant, according to the present teachings may include one or more of those based on polybutenyl succinic acid imide, polybutenyl succinic acid amide, benzylamine, succinic acid ester, succinic acid ester-amide, or a boron derivative thereof. The ashless dispersant may be incorporated normally at 0.05 weight % to 7 weight % of the total weight of the lubricating fluid.

Examples of the detergent, including metallic detergent, according to the present teachings may include one or more of those containing a sulfonate, phenate, salicylate, and phosphate of calcium, phosphate of magnesium, phosphate of barium, or the like. It may be optionally selected from perbasic, basic, neutral salts, and so forth of different acid value. The metallic detergent is optionally incorporated at 0.05 weight % to 5 weight % of the total weight of the lubricating fluid.

Examples of the defoamer according to the present teachings may include one or more of polydimethylsilicone, trifluoropropylmethylsilicone, colloidal silica, a polyalkyl acrylate, a polyalkylmethacrylate, an alcohol ethoxy/propoxylate, a fatty acid ethoxy/propoxylate, and a sorbitan partial fatty acid ester. The defoamer may be incorporated normally at 10 to 100 mg/l.

Examples of the antioxidant according to the present teachings may include one or more of amine-based antioxidants, e.g., alkylated diphenylamine, phenyl-α-naphtylamine and alkylated phenyl-x-naphtylamine: phenol-based ones, e.g., 2,6-di-t-butyl phenol, 4,4′-methylenebis-(2,6-di-tbutyl phenol) and isooctyl-3-(3,5-di-t-butyl-4-hydroxyphenyl) propionate; and/or -11-sulfur-based antioxidants, e.g., dilauryl-3,3′-thiodipropionate; and zinc dithiophosphate. The antioxidant may be incorporated normally at 0.05 weight % to 5 weight % of the total weight of the lubricating fluid.

Examples of the rust inhibitor according to the present teachings may include one or more of a fatty acid, alkenylsuccinic acid half ester, fatty acid soap, alkylsulfonate, polyhydric alcohol/fatty acid ester, fatty acid amine, oxidized paraffin, and alkylpolyoxyethylene ether. The rust inhibitor may be incorporated normally at 0 weight % to 37 weight % of the total weight of the lubricating fluid.

Examples of the friction modifier according to the present teachings may include one or more of an organomolybdenum-based compound, higher alcohols such as oleyl alcohol and stearyl alcohol: fatty acids such as oleic acid and stearic acid: esters such as oleyl glycerin ester, steryl glycerin ester, and lauryl glycerin ester: amides such as lauryl amide, oleyl amide, and stearyl amide; amines such as laurylamine, oleylamine, stearylamine, and an alkyldiethanolamine; and ethers such as lauryl glycerin ether and oleyl glycerin ether, oil/fat, amine, sulfided ester, phosphoric acid ester, acid phosphoric acid ester, acid phosphorous acid ester and amine salt of phosphoric acid ester. The friction modifier may be incorporated normally at 0.05 weight %. to 5 weight % of the total weight of the lubricating fluid.

Examples of the extreme pressure additive according to the present teachings may include one or more of organic sulfur, phosphorus or chlorine compounds, including sulfur-phosphorus and sulfur-phosphorusboron compounds, which chemically react with the metal surface under high pressure conditions.

Examples of the anti-wear additive according to the present teachings may include one or more of zinc dithiophosphate, zinc dialkyl dithio phosphate, tricresyl phosphate, halocarbons (chlorinated paraffins), glycerol mono oleate, and stearic acid.

Examples of the corrosion inhibitor according to the present teachings may include zinc dithiophosphates.

Examples of the pour point depressant according to the present teachings may include one or more of ethylene/vinyl acetate copolymer, condensate of chlorinated paraffin and naphthalene, condensate of chlorinated paraffin and phenol, polymethacrylate, polyalkyl styrene, and so forth. The pour point depressant may be incorporated normally at 0.1 weight % to 10 weight % of the total weight of the lubricating fluid.

In addition, solubilizing agents (i.e., co-solvents) that are used to dissolve polar additives in usually less polar or non-polar base oils may be included according to the present teachings.

A total content of additive(s) in the lubricating fluid composition of the present teachings is not limited. However, one or more additives (including the above-described solubilizing agent) may be incorporated at a concentration of about 1 weight % to 30 weight % of the total weight of the lubricating fluid, preferably 2 weight % to 15 weight % of the total weight of the lubricating fluid.

For example, provided herein is a high traction fluid composition comprising a base oil mixture as provided herein, and one or more additives selected from the group consisting of dispersants, detergents, defoamers, antioxidants, rust inhibitors, friction modifiers, corrosion inhibitors, extreme pressure additives, anti-wear additives, pour point depressants, and combinations thereof, wherein the composition comprises said one or more additives in a concentration of from about 1 weight percent to about 30 weight percent based upon the total weight of the composition.

The high traction base oils and compositions provided herein are useful in a wide variety of industrial and automotive applications. For example, the provided base oils and compositions are useful in many applications involving elasto-hydrodynamic lubrication, such as gear systems, ball and roller bearings, rolling assemblies, and belt drives. The provided base oils and compositions are especially useful as lubricants in transmission systems, such as industrial gear-less Planetroll type systems, as well as in newer generations of automotive transmission systems, particularly those comprising continuously variable transmissions (CVT). Other non-limiting examples of suitable applications for the provided base oils and compositions include use in machinery as engine oils, compressor oils, gear oils, and piston oils: as hydraulic, brake, and gear box fluids; as lubricants in turbines, compressors, and vacuum pumps; and in metal working and machining operations.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

1. A lubricating fluid comprising:
a Hydrogenated Linear AMS Dimer of 2,4-dicyclohexyl-2-methylpentane with a structure of: 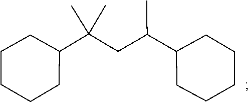 [SMILES: CC(CC(C)(C)C1CCCCC1)C1CCCCC1]
and
a Hydrogenated Linear AMS Dimer isomer of 1,4-dicyclohexyl-4-methylpentane with a structure of:

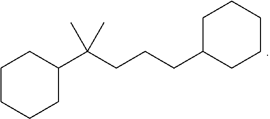 [SMILES: CC(C)(CCCC1CCCCC1)C1CCCCC1]

2. The lubricating fluid of claim 1, wherein the lubricating fluid contains:
from about 89.8 weight % to about 55.4 weight % of the Hydrogenated Linear AMS Dimer; and
from about 8.5 weight % to about 42.9 weight % of the Hydrogenated Linear AMS Dimer isomer.

3. The lubricating fluid of claim 2, wherein the lubricating fluid contains:
from about 87.5 weight % to about 59.3 weight % of the Hydrogenated Linear AMS Dimer; and
from about 10.8 weight % to about 39.1 weight % of the Hydrogenated Linear AMS Dimer isomer.

4. The lubricating fluid of claim 3, wherein the lubricating fluid contains:
from about 81.9 weight % to about 69.4 weight % of the Hydrogenated Linear AMS Dimer; and
from about 16.4 weight % to about 28.9 weight % of the Hydrogenated Linear AMS Dimer isomer.

5. The lubricating fluid of claim 1, wherein a ratio of the Hydrogenated Linear AMS Dimer to the Hydrogenated Linear AMS Dimer isomer is in a range of from about 10:1 to about 1.3:1.

6. The lubricating fluid of claim 5, wherein a ratio of the Hydrogenated Linear AMS Dimer to the Hydrogenated Linear AMS Dimer isomer is in a range of from about 8:1 to about 1.5:1.

7. The lubricating fluid of claim 6, wherein a ratio of the Hydrogenated Linear AMS Dimer to the Hydrogenated Linear AMS Dimer isomer is in a range of from about 5:1 to about 2.4:1.

8. A base oil comprising:
a Hydrogenated Linear AMS Dimer of 2,4-dicyclohexyl-2-methylpentane with a structure of: 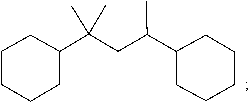 [SMILES: CC(CC(C)(C)C1CCCCC1)C1CCCCC1]
and
a Hydrogenated Linear AMS Dimer isomer of 1,4-dicyclohexyl-4-methylpentane with a structure of:

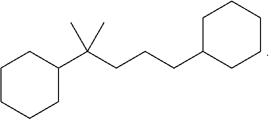 [SMILES: CC(C)(CCCC1CCCCC1)C1CCCCC1]

9. The base oil of claim 8, wherein the lubricating fluid contains:
about 89.8 weight % to about 55.4 weight % of the Hydrogenated Linear AMS Dimer; and
about 8.5 weight % to about 42.9 weight % of the Hydrogenated Linear AMS Dimer isomer.

10. The base oil of claim 9, wherein the lubricating fluid contains:
from about 87.5 weight % to about 59.3 weight % of the Hydrogenated Linear AMS Dimer; and
from about 10.8 weight % to about 39.1 weight % of the Hydrogenated Linear AMS Dimer isomer.

11. The base oil of claim 10, wherein the lubricating fluid contains:
from about 81.9 weight % to about 69.4 weight % of the Hydrogenated Linear AMS Dimer; and
from about 16.4 weight % to about 28.9 weight % of the Hydrogenated Linear AMS Dimer isomer.

12. The base oil of claim 8, wherein a ratio of the Hydrogenated Linear AMS Dimer to the Hydrogenated Linear AMS Dimer isomer is in a range of from about 10:1 to about 1.3:1.

13. The base oil of claim 12, wherein a ratio of the Hydrogenated Linear AMS Dimer to the Hydrogenated Linear AMS Dimer isomer is in a range of from about 8:1 to about 1.5:1.

14. The base oil of claim 13, wherein a ratio of the Hydrogenated Linear AMS Dimer to the Hydrogenated Linear AMS Dimer isomer is in a range of from about 5:1 to about 2.4:1.

15. A high traction fluid composition comprising:
(a) the base oil of claim 8; and
(b) one or more additives selected from the group consisting of dispersants, detergents, defoamers, antioxidants, rust inhibitors, friction modifiers, corrosion inhibitors, extreme pressure additives, anti-wear additives, pour point depressants, and combinations thereof
wherein the composition comprises said one or more additives in a concentration of from about 1 weight percent to about 30 weight percent based upon the total weight of the composition.

16. The high traction fluid composition of claim 15 comprising at least one dispersant selected from the group consisting of polybutenyl succinic acid imide, polybutenyl succinic acid amide, benzylamine, succinic acid ester, and succinic acid ester-amide.

17. The high traction fluid composition of claim 15 comprising at least one dispersant in a concentration of from about 0.05 weight percent to about 7 weight percent based upon the total weight of the composition.

18. The high traction fluid composition of claim 15 comprising at least one metallic detergent.

19. The high traction fluid composition of claim 18 comprising a metallic detergent selected from the group consisting of a sulfonate, phenate, salicylate, or phosphate of calcium, a phosphate of magnesium, and a phosphate of barium.

20. The high traction fluid composition of claim 15 comprising at least one metallic detergent in a concentration of from about 0.05 weight percent to about 5 weight percent based upon the total weight of the composition.

21. The high traction fluid composition of claim 15 comprising at least one defoamer selected from the group consisting of polydimethylsilicone, trifluoropropylmethylsilicone, colloidal silica, a polyalkyl acrylate, a polyalkylmethacrylate, an alcohol ethoxy/propoxylate, a fatty acid ethoxy/propoxylate, and a sorbitan partial fatty acid ester.

22. The high traction fluid composition of claim 15 comprising at least one defoamer in a concentration of from about 10 mg/L to about 100 mg/L.

23. The high traction fluid composition of claim 15 comprising at least one antioxidant selected from the group consisting of amine-based antioxidants, phenol-based antioxidants, and sulfur-based antioxidants.

24. The high traction fluid composition of claim 23 comprising at least one amine-based antioxidant selected from the group consisting of alkylated diphenylamine, phenyl-α-naphtylamine and alkylated phenyl-x-naphtylamine.

25. The high traction fluid composition of claim 23 comprising at least one phenol-based antioxidant selected from the group consisting of 2,6-di-t-butyl phenol, 4,4′-methylenebis-(2,6-di-tbutyl phenol) and isooctyl-3-(3,5-di-t-butyl-4-hydroxyphenyl)propionate.

26. The high traction fluid composition of claim 23 comprising at least one sulfur-based antioxidant selected from the group consisting of dilauryl-3,3′-thiodipropionate and zinc dithiophosphate.

27. The high traction fluid composition of claim 15 comprising at least one rust inhibitor selected from the group consisting of a fatty acid, alkenylsuccinic acid half ester, fatty acid soap, alkylsulfonate, polyhydric alcohol/fatty acid ester, fatty acid amine, oxidized paraffin, and alkylpolyoxyethylene ether.

28. The high traction fluid composition of claim 15 comprising at least one rust inhibitor in a concentration of up to about 37 weight percent based upon the total weight of the composition.

29. The high traction fluid composition of claim 15 comprising at least one friction modifier selected from the group consisting of organomolybdenum-based compounds, oleyl alcohol, stearyl alcohol, oleic acid, stearic acid, oleyl glycerin ester, steryl glycerin ester, lauryl glycerin ester, lauryl amide, oleyl amide, stearyl amide, laurylamine, oleylamine, stearylamine, alkyldiethanolamine; lauryl glycerin ether, oleyl glycerin ether, sulfided esters, phosphoric acid esters, acid phosphoric acid esters, acid phosphorous acid esters and amine salts of phosphoric acid esters.

30. The high traction fluid composition of claim 15 comprising at least one rust inhibitor in a concentration of from about 0.05 weight percent to about 5 weight percent based upon the total weight of the composition.

31. The high traction fluid composition of claim 15 comprising at least one extreme pressure additive selected from the group consisting of organic sulfur compounds, organic phosphorus compounds, and organic chlorine compounds.

32. The high traction fluid composition of claim 15 comprising at least one extreme pressure additive in a concentration of from about 0.05 weight percent to about 5 weight percent based upon the total weight of the composition.

33. The high traction fluid composition of claim 15 comprising at least one anti-wear additive selected from the group consisting of zinc dithiophosphate, zinc dialkyl dithio phosphate, tricresyl phosphate, halocarbons (chlorinated paraffins), glycerol mono oleate, and stearic acid.

34. The high traction fluid composition of claim 15 comprising at least one anti-wear additive in a concentration of from about 0.05 weight percent to about 5 weight percent based upon the total weight of the composition.

35. The high traction fluid composition of claim 15 comprising at least one pour point depressant selected from the group consisting of a ethylene/vinyl acetate copolymer, a condensate of chlorinated paraffin and naphthalene, a condensate of chlorinated paraffin and phenol, polymethacrylate, and polyalkyl styrene.

36. The high traction fluid composition of claim 15 comprising at least one pour point depressant in a concentration of from about 0.1 weight percent to about 10 weight percent based upon the total weight of the composition.
